(19) United States
(12) Patent Application Publication
(10) Pub. No.: US 2005/0251066 A1
(43) Pub. Date: Nov. 10, 2005

(54) Combination apparatus for recording the movements of the lower jaw in relation to the skull and for transferring upper jaw models into an articulator correctly with respect to the skull

(75) Inventor: Heinz Mack, Munchen (DE)

Correspondence Address: STITES & HARBISON PLLC, 1199 NORTH FAIRFAX STREET, SUITE 900, ALEXANDRIA, VA, 22314, US

(73) Assignee: SAM PRAZISIONSTECHNIK GMBH

(21) Appl. No.: 11/086,339

(22) Filed: Mar. 23, 2005

(30) Foreign Application Priority Data
Mar. 26, 2004 (DE) 10 2004 015 383.3

(51) Int. Cl. A61B 5/103

(52) U.S. Cl. 600/590

(57) ABSTRACT

A combination apparatus for recording the movements of the lower jaw in relation to the skull and for transferring upper jaw models into an articulator correctly with respect to the skull is described, which apparatus is principally composed of (a) an upper part which is placed on the head at the level of the nasion and of the temples and is principally composed of a U-shaped frame with two side parts and a transverse part, which frame is adapted to the particular head shape and is fixed on the head by means of securing and retaining devices and, on its transverse part, has a nasion support and a device for receiving a bite-fork carrier with bite fork, and which, on the two side parts, is equipped with in each case an upper sensor support device carrying at least three upper transmitting or receiving devices which transmit or receive electronic, optical or acoustic signals and which are connected via corresponding lines to the data processing equipment or other data transmission devices, and (b) a lower part secured on the lower jaw and composed principally of bite fork, reference pins and a lower sensor support device carrying the receiving and transmitting devices which correspond to the upper transmitting or receiving devices in the upper part and which are likewise connected via corresponding lines to the data processing equipment or other data transmission devices.

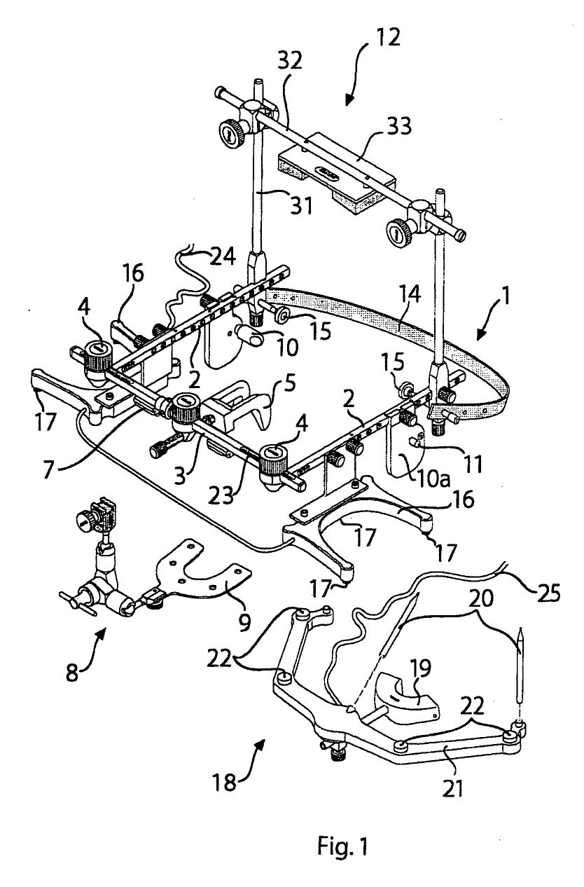

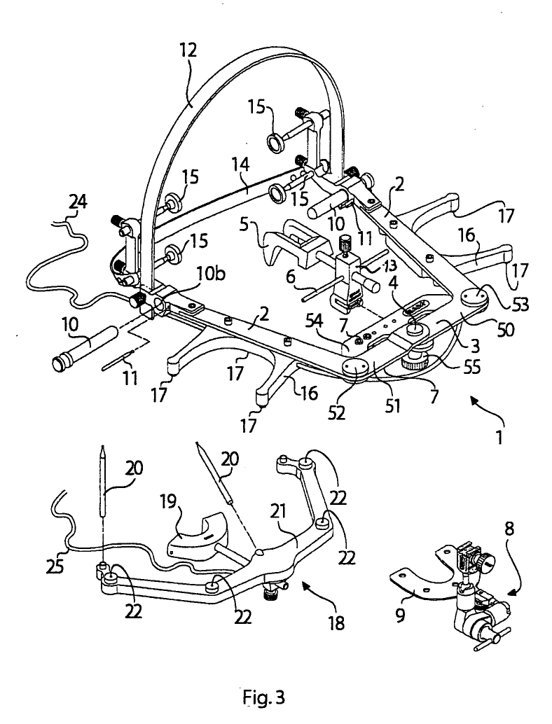

The nasion support is preferably designed as shown in FIGS. 1 and 3.

The present invention relates to a combination apparatus for recording the movements of the lower jaw in relation to the skull and for transferring upper jaw models into an articulator correctly with respect to the skull.

The previously disclosed apparatuses for recording the movements of the lower jaw in relation to the skull are used to exactly determine the movements of the joints of the lower jaw for diagnostic purposes, i.e. to allow conclusions to be drawn regarding the pathological state of the joints of the lower jaw. This involves detecting abnormal patterns of movement of the jaw joints through the influence of musculature and/or articular disk, in order to establish a treatment plan on the basis of the diagnosis, said patterns of movement being measured directly in the joint area and being recorded and then converted numerically and graphically by computer to the respectively desired values.

An apparatus of this kind for recording the movements of the lower jaw is sold under the protected brand name of AXIOGRAPH and is described in the applicant's German patent specification DE 44 41 991 C2 (Mack-12), to which reference is hereby expressly made for purposes of disclosure. It is principally composed of:
(a) an adjustable upper recording bow which is secured on the nasion and in the area of the temples, with
   a nasion support,
   measuring/recording plates,
   upper reference indicator,
   positioning devices with ear inserts mounted thereon and retaining pins for the positioning of the lower recording bow, which positioning devices can later be replaced by the measuring/recording plates,
   a vertical frame arranged perpendicularly and with support columns which in the upper area are connected to an adjustable transverse bar, on which the head rest is secured, and which in the lower area are provided with laterally adjustable and lockable side spacers,
   a rear U-shaped auxiliary frame which consists of transverse profile and lockable longitudinal profiles and which, for the purpose of parallelization of the side arms, can be adjusted to the front spacing of the side arms and can be secured on the side arms, and
   a nasion support which can be finely adjusted in the longitudinal direction and is laterally displaceable on the front transverse bar, and
   a band for the back of the neck, and
(b) a lower recording bow which is connected to the movable lower jaw via a tray or a clamping device, where
   the lower recording bow has, on both side arms secured on the transverse bar, devices into which a recording and measuring device is introduced which permits continuous recording of the movements in all 3 planes, i.e. permits recording of the vertical and horizontal movements and at the same time measuring of the axial displacements of the horizontal movements mechanically, electronically, acoustically or optically and/or permits recording via an auxiliary unit,
   the lower recording bow, for positioning on the retaining pins of the positioning device of the upper recording bow, has retainer bushings which at the rear end are guided through the securing devices and are locked by means of the locking screws and are fitted onto the retaining pins, and where
   after securing of the tray or securing of a corresponding clamping device on the lower jaw, and after positioning of the upper recording bow and of the lower recording bow, the positioning devices are replaced by the measuring/recording plates and, on the lower recording bow, the retainer bushings are replaced by the recording and measuring devices.

A disadvantage of this apparatus was felt to be the fact that the lower recording bow has to be positioned exactly with respect to the upper recording bow, for which purpose, as the above observations show, a relatively complex mechanical arrangement is needed. It is only by such precise positioning that the movements of the joints of the lower jaw can be detected directly in the joint area with the required precision.

Therefore, the object of the present invention is to configure such apparatuses (axiographs) in such a way that all the movements of the lower jaw can be detected without such great mechanical complexity, and also in such a way that the upper recording bow can satisfy the function of a customary anatomical transfer bow.

A customary anatomical transfer bow is described, for example, in the applicant's German patent specification DE 30 32 913 C2 (Mack-3), in conjunction with the applicant's German laid-open specification DE 101 42 139 A1 (Mack-18), to which reference is made in full for reasons of disclosure.

The anatomical transfer bow is used to transfer the reference plane (which is formed by the imaginary linear connection of three anatomical reference points at the base of the patient's skull) from the patient to the articulator in order in this way to position the jaw models, produced by impressions, in the articulator (simulator) in a manner which is correct in relation to the skull, i.e. with the correct coordinates in respect of the skull. Only in this way is it possible to correctly simulate the functions of the movable lower jaw in relation to the upper jaw which is fixed relative to the lower jaw. The three anatomical reference points are defined by the two external auditory canals and the nasion.

Models mounted in the articulator with the correct orientation with respect to the skull are thus likewise an important tool in diagnosis and therapy. The correct spatial relationship of the models to the reference plane is a prerequisite for setting the occlusion plane, the condyle path angles, according to statistical mean values and in particular for mounting with centric register.

The reference points for the Frankfort horizontal reference plane are the highest points of the two external auditory canals (the poria) and the lowest point of the bony framework of the orbit (the orbitale).

The bite-fork carrier remains symmetrically positioned by means of optimized pivot points of the pivotable side arms of the anatomical transfer bow. The bite-fork carrier preferably consists of a three-dimensionally adjustable mechanical connection of rods and clamps, in particular of a cardan joint (cf. DE 195 30 536 A1 (Mack-15) and DE 101 14 798 A1 (Mack-17).

The nasion support, lying on the bridge of the nose serving as reference point, maintains the distance from the reference plane and ensures that the anatomical transfer bow lies at the correct spacing from the orbitale of the patient. By means of the two ear inserts (ear rods) located on the transfer bow, the anatomical transfer bow is oriented on the porion reference points (porion is the highest point of the external auditory canal).

For esthetic reasons, it is a requirement in dentistry that the line of the cutting edges of the teeth of the upper jaw lies parallel to the line connecting the midpoints of the pupils (interpupillary line) and that the upper jaw model obtained from taking an impression of the upper jaw is transferred in the same (correctly coordinated) position from the skull to the articulator.

For this reason, the anatomical transfer bow preferably also has a leveling device for aligning it with the interpupillary line.

With this leveling device (leveling aid) additionally arranged on the anatomical transfer bow, it is possible for the transfer bow, which is fitted in the two external auditory canals and lies on the nasion, to be readjusted in a simple manner in parallel with reference to the interpupillary line (precision adjustment).

This leveling device is preferably a sighting pin which protrudes, parallel to the central part, on both sides of the mounting part of the nose support.

In addition to the aforementioned object, it is therefore also an object of the present invention to equip a customary anatomical transfer bow in such a way that it can also serve as an apparatus for recording the movements of the lower jaw in relation to the skull.

According to the present invention, the two aforementioned objects are achieved by the fact that the recording of all of the movements of the lower jaw no longer takes place just in the joint area, but instead the three-dimensional movements of the whole of the lower jaw are recorded in relation to the skull. According to the present invention, this is achieved by the fact that the two side arms of the upper recording bow (upper part) of the axiograph or transfer bow are each equipped with an upper sensor support device which carries at least three transmitting or receiving devices for transmitting or receiving electronic, optical or acoustic signals, and the lower recording bow of the AXIOGRAPH® or the bite-fork carrier with bite fork of the anatomical transfer bow is replaced by a lower part which is provided with a tray (with which the lower part is secured on the lower jaw) and on which a lower sensor support device is mounted, which device carries the receiving or transmitting devices which correspond to the transmitting or receiving devices in the upper part and which are connected via corresponding lines to the data processing equipment or via other data transmission devices, e.g. by radio.

With such an arrangement, it is possible to dispense with the mechanically very complex positioning and coupling of the lower recording bow to the upper recording bow. In this way, with the recording bow (upper part) carrying the upper sensors and the lower part carrying sensors, it is possible to obtain a contactless electronic, optical or acoustic recording device which can also be designated as a “sensor axiograph”. Correspondingly, the anatomical transfer bow is incorporated into an upper part with corresponding sensor support devices which then, like the above-described lower part supporting the lower sensor support device, can be used as “sensor axiograph” for three-dimensional recording of all movements of the lower jaw.

Since not only have the sensor support devices been mounted on the upper recording bow (upper part) of the axiograph, but the front area also has a device for receiving a bite-fork carrier with bite fork, the upper part of the novel “sensor axiograph”, by application of the bite-fork carrier with the bite fork, can assume the function of an anatomical transfer bow.

The subject of the present invention is therefore a combination apparatus for recording the movements of the lower jaw in relation to the skull and for transferring upper jaw models into an articulator correctly with respect to the skull, which apparatus is principally composed of
(a) an upper part which is placed on the head at the level of the nasion and of the temples and is principally composed of a U-shaped frame with the two side parts and the transverse part, which frame can be adapted to the particular head shape and, on its transverse part, has adjustment devices, a nasion support, if appropriate with leveling device, and a device for receiving a bite-fork carrier with bite fork, and which, in the end areas of the two side parts, is further equipped with devices for the ear inserts, having axial pins, with a securing device extending across the skull cap, with a retaining device resting on the back of the neck, and with laterally adjustable and lockable side spacers, and which, on the two side parts, is finally equipped with in each case an upper sensor support device carrying at least three upper transmitting or receiving devices which transmit or receive electronic, optical or acoustic signals and which are connected via corresponding lines or other data transmission devices to the data processing equipment, and
(b) a lower part composed of bite fork, reference pins and a lower sensor support device carrying the receiving or transmitting devices which correspond to the upper transmitting or receiving devices in the upper part and which are likewise connected via corresponding lines or other data transmission devices to the data processing equipment.

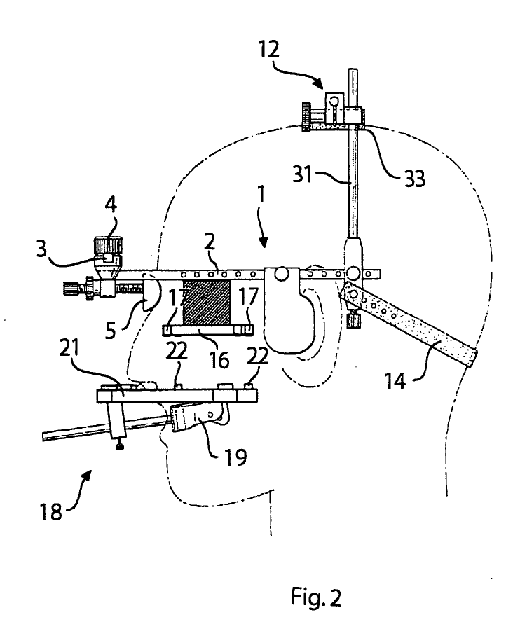

A preferred embodiment of the combination apparatus according to the invention, which derives from the upper recording bow of the axiograph, is shown in FIGS. 1 and 2 and is distinguished by the fact that the two side arms of the U-shaped frame of the upper part are attached so as to be displaceable parallel to one another on a front transverse bar, and the ends of the side arms, for the purpose of positioning the upper part on the head in a manner secure against displacement, are additionally connected to a perpendicularly attached vertical frame whose support columns are connected in the upper area to an adjustable transverse bar on which the head rest is secured, and, in the lower area, they are provided with laterally adjustable and lockable side spacers.

A further preferred embodiment according to the present invention, which derives from the anatomical transfer bow, is shown in FIGS. 3 through 5 and is distinguished by the fact that the U-shaped frame of the upper part of the combination apparatus is principally composed of two L-shaped side arms which in the front area are connected spreadably like scissors via pivot bearings to a base plate at two opposite peripheral pivot points and can be locked in the overlap area of the ends of the transverse branches of the L-shaped side arms, and
(a) the pivot bearings are arranged in the respective L-bend of the L-shaped side arms, and
(b) the base plate has, in the middle between the pivot bearings, an oblong hole whose longitudinal edges extend parallel to the longitudinal axis of the upper part and through which extends the locking device which passes through the free ends of the transverse branches in the overlap area pivotably, but secure against displacement.

According to a further preferred embodiment of the present invention, the nasion support 5 is preferably designed as shown in FIGS. 1 and 3, i.e. instead of the hitherto customary nasion roller, the nasion support 5 has the form of two jibs which extend parallel to one another and which, at their ends, are curved and drift away from one another from the top downward and in this way form a practically immovable support on the nasion. In this way, it is additionally possible to continue the customary profile scanning from the forehead across the nasion, which is not possible in the previous nasion supports.

The invention is explained with reference to FIGS. 1 through 5, which are self-explanatory, but without limiting the invention to these. All the details shown in the figures belong to the disclosure of the invention, as also do the explanations in the already cited protection rights belonging to the applicant.

The reference numbers used in FIGS. 1 through 5 have the following meanings:
1 upper part
2 side parts of the U-shaped frame
3 transverse part of the U-shaped frame
4 adjustment devices
5 nasion support
6 leveling device
7 device for receiving the bite-fork carrier
8 bite-fork carrier
9 bite fork
10 ear inserts
10a support device for ear insert
10b bore for ear insert
11 axial pin
12 securing device extending across skull cap
13 carrier for the nasion support 5
14 retaining device resting on back of neck
15 lockable side spacers
16 support device for upper sensors
17 upper transmitting or receiving device
18 lower part
19 tray for securing on the lower jaw
20 reference pins
21 support device for lower sensors
22 lower transmitting or receiving device
23 transverse bar of the U-shaped frame
24,25 lines to the data processing equipment
31 support column of the vertical frame
32 transverse bar of the vertical frame
33 head rest
50,51 transverse branches of the L-shaped side parts 2
52,53 pivot bearings
54 base plate
55 locking device

FIG. 1 shows a perspective view of the combination apparatus according to the invention which, as upper part 1, has the upper recording bow of the apparatus for recording the movements of the lower jaw according to the conventional axiograph, with upper part 1, lower part 18, and bite-fork carrier 8 with bite fork 9.

Upper part 1 and lower part 18 are equipped with the upper and lower sensor support devices 16, 21, which respectively carry the transmitting or receiving devices 17, 22. After the upper part 1 is placed on the head, the lower part 18 is secured on the lower jaw by means of the bite fork 19.

Both the upper transmitting or receiving devices 17 and the lower transmitting or receiving devices 22 are connected to the data processing equipment by way of lines 24 and 25.

The transmitting or receiving devices 17, 22 are designed such that electronic, optical or acoustic signals can be transmitted or received with them. According to a preferred embodiment of the present invention, an ultrasound system is used, the transmitting devices then being arranged in the upper part 1 and the receiving devices being arranged in the lower part 18. However, the arrangement can equally well be reversed, so that the transmitting devices are located in the lower part 18 and the receiving devices in the upper part 1. The ultrasound system operates in a contactless manner and parallel to the porion-orbitale reference plane=Frankfort horizontal or axis-orbital plane and ensures a correct and coordinate-accurate measurement of the joint trajectories and the paths of the front teeth. The electronics compare the transit time measurements of the sensors. In the same way as in satellite navigation, the position and the trajectory of the movements of the lower jaw can be calculated therefrom by means of the data processing equipment.

The data processing equipment creates a clear relationship both for patients and also for different treatment providers or projects. The import and export of data out of and into other systems is possible.

If the upper part 1 is to serve as anatomical transfer bow, the bite-fork carrier 8 with the bite fork 9 is secured on the device 7 which is located on the upper part 1 in the transverse area 3 and which receives the bite-fork carrier.

FIG. 2 shows, in a diagrammatic representation, the apparatus according to the invention on a patient's head. The figure clearly shows the upper part 1, which corresponds to the upper recording bow of the axiograph, with its side part 2, its vertical frame with the support columns 31, the transverse bar 32 and the head rest 33. It also shows, on the front side of the upper part 1, the adjustable nasion support 5 which can be finely adjusted by means of a spindle. At the lower end of the support column 31, the figure shows the retaining device 14 (e.g. rubber band) which is placed resting on the back of the neck and which is guided round the back of the patient's head to the other side part. In this way, the upper part 1, which rests on the nose by way of the nasion support 5, acquires a firm fit on the head with the spacers 15 and the head rest 33. The upper sensor support device 16 with the upper transmitting or receiving devices 17 can be clearly seen on this upper part 1.

The figure also shows the lower part 18 which is secured on the lower jaw by means of tray 19 and is provided with the lower sensor support device which carries the lower transmitting or receiving devices 22 corresponding to the upper transmitting or receiving devices 17.

FIG. 3 shows the combination apparatus according to the invention as claimed in claim 3, in which the upper part 1 derives from the anatomical transfer bow, composed of the upper part 1, the lower part 18 and the bite-fork carrier 8 with bite fork 9.

As in the embodiment according to FIG. 1, upper part 1 and lower part 18 are equipped with upper and lower sensor support devices 16, 21 which carry the transmitting or receiving devices 17, 22. After the upper part 1 is placed on the head, the lower part 18 is secured on the lower jaw by means of the tray 19.

The ultrasound system is preferably used in this embodiment too.

If the upper part 1 is to serve as anatomical transfer bow, the bite-fork carrier 8 with bite fork 9 is secured on the device 7 which is located on the upper part 1 in the transverse area 3 and which receives the bite-fork carrier.

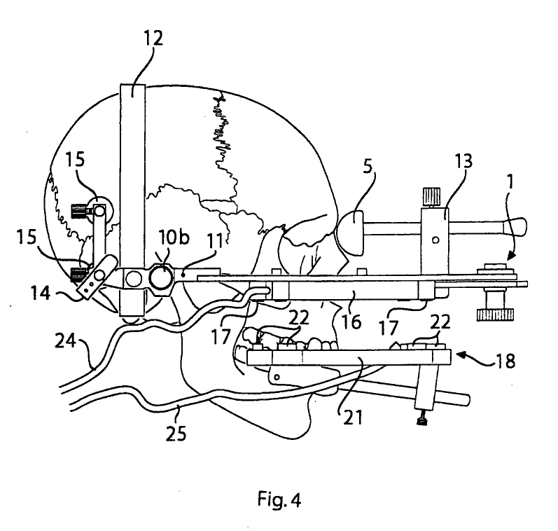

FIG. 4 shows a diagrammatic side view of a human skull fitted with the combination apparatus composed of upper part 1 and lower part 18 according to FIG. 3, and with the corresponding reference numbers whose meaning is indicated above, so that no further explanation of FIG. 4 is needed.

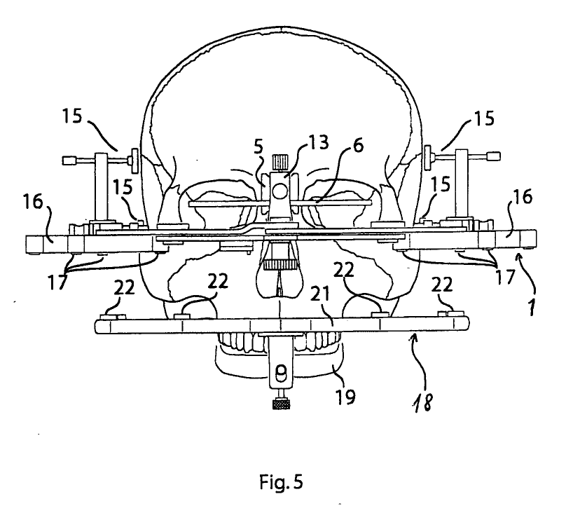

FIG. 5 shows a diagrammatic front view of a human skull fitted with the combination apparatus composed of upper part 1 and lower part 18 according to FIG. 3, and with the corresponding reference numbers whose meaning is indicated above, so that no further explanation of FIG. 5 is needed.

1. A combination apparatus for recording the movements of the lower jaw in relation to the skull and for transferring upper jaw models into an articulator correctly with respect to the skull, which apparatus is principally composed of
(a) an upper part (1) which is placed on the heat at the level of the nasion and of the temples and is principally composed of a U-shpaed frame with the two side parts (2) and the traverse part (3), which frame can be adapted o the particular head shape and, on its traverse part (3), has adjustment devices (4), a nasion support (5), if appropriate with level device (6), and a device (7) for receiving a bite-fork carrier (8) with bite fork (9), and which, in the end areas of the two side parts (2), is further equipped with devices (10a, 10b) for the ear inserts (10), having axial pins (11), with a securing device (12) extending across the skull cap, with a retaining device 914) resting on the back of the neck, and with laterally adjustable and lockable side spacers (15), and which, on the two side parts (2), is finally equipped with in each case an upper sensor support device (16) carrying at least three upper transmitting or receiving devices (17) which transmit or receive electronic, optical or acoustic signals and which are connected via corresponding lines (24) to the data processing equipment, and
(b) a lower part (18) composed of bite fork (19), references pins (20) and a lower sensor support device (21) carrying the receiving or transmitting devices (22) which correspond to the upper transmitting or receiving devices (17) in the upper part (1) and which are likewise connected via corresponding lines (25) to the data processing equipment.

2. The combination apparatus as claimed in claim 1, and in accordance with FIGS. 1 and 2, wherein the two side parts (2) of the U-shaped frame of the upper part (1) are attached so as to be displaceable parallel to one another on the transverse part (3), and the side parts (2), for the purpose of positioning the upper part (1) on the head in a manner secure against displacement, are additionally connected to a perpendicularly arranged vertical frame which extends across the skull cap and whose support columns (31) are connected in the upper area to an adjustable transverse bar (32) on which the head rest (33) is secured, and, in the lower area, they are provided with laterally adjustable and lockable side spacers (15).

3. The combination apparatus as claimed in claim 1, and in accordance with FIGS. 3 and 4, wherein the U-shaped frame of the upper part (1) is principally composed of two L-shaped side parts (2) with the transverse branches (50, 51) which, in the transverse part (3) formed by the transverse branches (50, 51) are connected spreadably like scissors via pivot bearings (52, 53) to a base plate (54) at two opposite peripheral pivot points and can be locked in the overlap area of the ends of the transverse branches (50, 51) of the L-shaped side parts (2), and
(a) the pivot bearings (52, 53) are arranged in the respective L-bend of the L-shaped side parts (2), and
the base plate (54) has, in the middle between the pivot bearings (52, 53), an oblong hole whose longitudinal edges extend parallel to the longitudinal axis of the upper part (1) and through which extends the locking device (55) which passes through the free ends of the transverse branches (50, 51) in the overlap area pivotably, but secure against displacement.

4. A nasion support, as is used in the apparatuses as claimed in claim 1, wherein, instead of the hitherto customary nasion roller, it has the for of two jibs which extend parallel to one another and which, at their ends, are curved and drift away from one another from the top downward.
